(19) United States
(12) Patent Application Publication
(10) Pub. No.: US 2005/0038510 A1
(43) Pub. Date: Feb. 17, 2005

(54) Multi-piece intraocular lens assembly and method of manufacturing same

(76) Inventors: Valdemar Portney, Tustin, CA (US); Donald A. Horvatich, Alta Loma, CA (US); Victor M. Gargia, Fontana, CA (US)

Correspondence Address: Howard R. Lambert, 5245 Gatewood Lane, Anaheim, CA, 92807, US

(21) Appl. No.: 10/897,619

(22) Filed: Jul. 24, 2004

Related U.S. Application Data
(60) Provisional application No. 60/494,924, filed on Aug. 13, 2003.

(51) Int. Cl. A61F 2/16; B29D 11/00

(52) U.S. Cl. 623/6.46; 623/909; 264/1.7

(57) ABSTRACT

A multi-piece intraocular lens (IOL) has an optic constructed of an elastomeric plastic and an opposing pair of attachment members (haptics) constructed of a rigid plastic material. The optic or haptics have one or more small, tear drop-shaped, inlet recesses formed into each from a peripheral edge region. The haptics or optic are formed having peripheral edge protuberances matching in size, shape and position corresponding optic or haptic inlet recesses. The protuberances dovetail into the corresponding inlet recesses and are cemented therein to lock the haptics and optic together. Methods for manufacturing a multi-piece IOL include forming the haptics from a modified haptic blank and the optic from a modified optic blank. After the haptic or optic protuberances are dovetailed and cemented into the optic or haptic inlet recesses, the haptics and optic are milled or lathed from the respective haptic and optic blanks to provide a completed multi-piece IOL.

### RELATED APPLICATION

This application is a utility application converted from provisional application Ser. No. 60/494,924, filed Aug. 13, 2003, such provisional application being incorporated herein in its entirety by specific reference.

### BAKGROUND OF THE INVENTION

1. Field of the Invention

The present invention relates generally to the field of ophthalmics, more particularly to intraocular lenses (IOLs) and still more particularly to multi-piece intraocular lens and associated manufacturing methods.

2. Background Discussion

Intraocular lenses (IOLs) are now commonly implanted in individual's eyes from which the natural crystalline lens has been removed (aphakic eyes), usually because cataracts have diminished vision in the eye, to restore vision in the eye. IOLs may alternatively be implanted in eyes from which the natural lens has not been removed (phakic eyes) to correct vision conditions such as myopia, hyperopia and astigmatism. In the case of aphakic eyes, IOLs are most commonly implanted in the posterior capsule from which the natural crystalline lens had been removed, now usually by a process in which the natural lens is first ultrasonically fragments and the particles aspirated from the eye—a process called phacoemulisification. In the case of phakic eyes, IOLs may be implanted in either the ocular posterior or anterior chambers.

IOLs comprise an optic having a typical diameter of about 5 mm and, with few exceptions, have two (or sometimes three or four) attachment elements, called haptics, attached to, or formed at, the optic periphery. The haptics secure the IOL in a patient's eye with the optical axis of the optic aligned with the optical axis of the eye. IOL optics may be constructed from a clear, rigid plastic, principally polymethyl methacrylate (PMMA), or are now preferably constructed from a clear, soft plastic, such as a silicone or acrylic material, which enables the optic to be elastically deformed (folded or rolled) for implanting into a patient's eye through a much smaller ocular incision than is required for implanting of a rigid optic. Regardless of optic material, the haptics of multi-piece IOLs are usually constructed from a polypropylene plastic material as a “springy” filament A common method of producing a multi-piece IOL involves attaching a pair of haptics to the optic by drilling holes in opposite regions of the optic edge and inserting the haptics into the drilled holes (Ref. U.S. Pat. No. 6,432,230; U.S. Pat. No. 6,252,312; U.S. Pat. No. 5,523,029 and U.S. Pat. No. 5,266,241). Different methods of loop-to-optic locking are used, including staking with a heated probe and gluing, with gluing being preferred to minimize potential damage to the optic from a heated probe. A principal downside of the gluing process is that the attachment portion of the haptics must closely confirm to the drilled holes at the optic edge, thus limiting the attachment to a rounded haptic shape.

Another method of multi-piece IOL fabrication involves molding the optic body around pre-fabricated haptics (Ref. U.S. Pat. No. 6,555,030; U.S. Pat. No. 6,159,242; U.S. Pat. No. 5,171,268 and U.S. Pat. No. 5,141,507). It is a challenge to use such a molding IOL fabrication method for complex shaped haptics requiring high accuracy of fabrication, due to a high temperature involved in the molding process.

In order to overcome difficulties of producing IOLs with a variety of haptic shapes, a method of multi-material for single-piece IOL has been disclosed (Ref. U.S. Pat. No. 5,326,506; U.S. Pat. No. 5,217,491; U.S. Pat. No. 5,211,662 and FRPN 2,779,944). This particular method disclosed involves making multi-material blanks though chemical bounding of the materials during the manufacturing process of molding and polymerization and then producing the IOL using milling and lathing operations. The corresponding haptics can only be produced from a blank as a single-piece IOL because the haptic to optic attachment involves a transition from one material to another to form a chemical bound. This method is limited to chemically similar materials for optic and haptics in order to create a transition from one to the other in the corresponding manufacturing process.

There are IOLs with more complex haptic shapes, such as IOLs with plate haptics, and anterior chamber IOLs for angle support or iris fixation placement. These IOLs can be made as multi-piece IOLs to manifest different characteristics of the optic and haptics. As mentioned above, it is often highly desirable to make the optic of an elastomeric material, such as a silicone or acrylic plastic, to enable optic folding or rolling and insertion into the eye through a small incision. On the other hand, the haptics provide optic fixation and/or support inside the eye, which requires different characteristics. It is thus desirable for the haptic to manifest a rigid property to maintain optic centration inside the eye or allowing fixation to the iris tissue in case of iris-fixated IOLs; this the haptics may be formed from a polymethyl methacrylate (PMMA) plastic material.

Thus, a multi-piece IOL with a complex haptic shape can be produced by milling and lathing operations where each haptic is attached to a separately fabricated optic. This involves the very tedious operation of drilling rounded holes in the side edges of the optic and forming a rounded pin at the haptic ends for insertion into the drilled holes.

Additional difficulty is maintaining orientational stability of a complex-shaped haptic upon its fixation into the optic. The haptic must maintain its orientation after its attachment to the optic in order to provide the desired optic position inside the eye. Thus, the attachment of haptics made of a complex shape into the optic and the corresponding manufacturing process present significant challenge which is addressed by the present invention.

### SUMMARY OF THE INVENTION

A multi-piece intraocular lens comprises an optic having an optical axis and a peripheral edge, the optic peripheral edge having at least one small inlet; and a haptic having an optic attachment region with at least one first small projection shaped to fit closely into the at least one optic small inlet recess through one of an optic anterior surface, an optic posterior surface and an optic peripheral edge. Preferably the optic small inlet recess has an enlarged region towards the optical axis so as to form a general keyhole shape.

The optic peripheral edge may have a first region with “n” first small inlet recesses and a second peripheral edge region with “m” second small inlet recesses, in which case, the haptic has an optic attachment region with “n” small projections shaped to dovetail into the optic “n” first small inlet recesses. A second haptic having an optic attachment region with “m” small projections shaped to dovetail into the optic “m” second small inlet recesses is then provided.

Preferably the numbers “n” and “m” are each selected from a group of numbers consisting of 1, 2 and 3 and the first and second optic peripheral edge regions are located diametrically opposite of each other. Also preferably the haptic is constructed from a material that is different from a material from which the optic is constructed.

In the alternative, a multi-piece intraocular lens comprises an optic having an optical axis and a peripheral edge having at least one first small projection and a haptic having an optic attachment region with at least one first small inlet recess shaped to receive the at least one optic small projection, the haptic inlet recess having an enlarged region so as to form a general keyhole shape.

The optic peripheral edge may have a first region with “n” first small projections and a second peripheral edge region with “m” second small projections; in which case, the haptic has an optic attachment region with “n” small inlet recesses shaped to closely receive the optic “n” first small projections a second haptic having an optic attachment region with “m” small inlet recesses shaped to closely receive the optic “m” second small projections is provided.

In particular, a multi-piece intraocular lens comprises an optic having an optical axis and a peripheral edge, the optic peripheral edge having a first region with “n” first small inlet recesses and a second region opposite to the first region having “n” second small inlet recesses. Each of the “n” first and second small inlet recesses have a general keyhole shape, the number “n” being selected from a group of numbers consisting of 1, 2 and 3. Included are a first haptic having an optic attachment region with “n” small projections shaped and located to dovetail into the optic “n” first small inlet recesses and a second haptic having an optic attachment region with “n” projections shaped and located to dovetail into the optic “n” second small inlet recesses.

Preferably, the first and second haptics are formed of a rigid plastic material and the optic is formed of an elastomeric plastic material.

A method of constructing a multi-piece intraocular lens comprises the steps of forming an optic of one material with a small inlet recess in a peripheral edge region and forming a positioning haptic of a different material with a small protrusion extending from an optic attachment region, the protrusion matching the shape of the optic small inlet recess. Included are the steps of inserting the haptic protrusion into the optic inlet recess through one of an optic anterior surface, an optic posterior surface and an optic peripheral edge so as to interlock the haptic and optic together and cementing the said haptic protrusion into said optic inlet recess.

The step of forming the haptic preferably includes forming the haptic of a rigid plastic material and the step of forming the optic preferably includes forming the optic of an elastomeric plastic material.

More particularly, a method of constructing a multi-piece intraocular lens comprises the steps of forming an optic of an elastomeric plastic material with a plurality of like small inlet recesses in each of two opposite peripheral edge regions and forming first and second positioning haptics of a hard plastic material, each with a plurality of small protrusions extending from an optic attachment region, the plurality of protrusions matching the shape and location of the optic small inlet recesses.

The method includes the further steps of inserting the first haptic plurality of small protrusions into the plurality of optic small inlet recesses in one optic peripheral edge region and the second haptic plurality of small protrusions into the plurality of optic small inlet recesses in the opposite optic peripheral edge region so as to dovetail interlock the haptics and optic together, and cementing the plurality of protrusions of the first and second haptics into corresponding pluralities of inlet recesses in the optic to form a composite, multi-piece intraocular lens.

An alternative method of constructing a multi-piece intraocular lens comprises the steps of forming a first modified blank of one material to be used for haptic fabrication with a central optic cut-out leaving at least one small protrusion for haptic to optic attachment and forming a second modified blank of another material to be used for optic fabrication with at least one peripheral edge small inlet recess at a location corresponding to a location of the protrusion in the first modified blank and matching the shape of the protrusion. The method includes the steps of assembling the first and second modified blanks into an interlocking, dual-material blank with the haptic small protrusion dovetailed into the optic small inlet recess; and fabricating a composite, multi-piece IOL from the dual-material blank by forming a haptic in the first modified blank.

Preferably the fabricating step includes cementing the haptic protrusion into the optic inlet recess and the steps of forming a haptic in the first modified blank includes forming the haptic of a rigid plastic material and the step of forming an optic in the second modified blank includes forming the optic of an elastomeric plastic material.

Another method of constructing a multi-piece intraocular lens comprises the steps of forming a first modified blank of one material to be used for haptic fabrication with a central optic cut-out leaving at least one small dovetail inlet recess in each of opposite peripheral optic cut-out regions for haptic to optic attachment, and forming a second modified blank of another material to be used for optic fabrication with at least one peripheral edge small protrusion in each of opposite peripheral edge regions at locations corresponding to location of the haptic inlet recesses in the first modified blank and matching the shape of the inlet recesses.

Included in the method are the steps of assembling the first and second modified blanks into a composite, dual-material blank with the optic protrusions interlockingly dovetailed and cemented into corresponding ones of the haptic inlet recesses, and fabricating a composite, multi-piece IOL from the dual-material blank by forming first and second haptics in the first modified blank and cementing the optic protrusions into the haptic inlet recesses.

Preferably, the step of forming the first and second haptics includes forming the haptics of a rigid plastic material and the step of forming the optic includes forming the optic of an elastomeric plastic material.

An variation method of constructing a multi-piece intraocular lens comprises the steps of forming a haptic from a first material, with at least one small inlet recess in an edge of the haptic; and forming an optic from a second material, with at least one peripheral edge small protuberance at a peripheral edge region, the at least one protuberance matching the at least one haptic small inlet recess in shape, number and position; and dovetailing and cementing the at least one optic protrusion into the at least one haptic inlet recess so as to provide a multi-piece intraocular lens.

It is preferred that the step of forming the haptic includes forming the haptic of a rigid plastic material and the step of forming the optic includes forming the optic of an elastomeric plastic material.

### BRIEF DESCRIPTION OF THE DRAWINGS

The present invention can be better understood by a consideration of the drawings, in which:

FIG. 1 is a partial plan view of a multi-piece intraocular lens (IOL) according to the present invention, showing an optic having a single keyhole-shaped, small inlet recess formed into a representative peripheral edge region and showing a representative attachment member (haptic) having a small, mating haptic-to-optic attachment protrusion dovetailed into the optic inlet recess;

FIG. 2 is a series of transverse cross sectional drawings taken along line 2-2 of FIG. 1, showing different haptic-to-optic attachments: FIG. 2A showing the optic small inlet recess formed fully into the optic peripheral edge; FIG. 2B showing a variation optic small inlet recess formed partially into the optic from an anterior surface of the optic; and FIG. 2C showing a second variation optic small inlet recess formed partially into the optic from a posterior surface of the optic, in each of such FIGS, the corresponding haptic small protrusion is shown received into the optic recess;

FIG. 3 is a partial plan view similar to FIG. 1 of a multi-piece intraocular lens (IOL), except showing an optic having two, side-by-side, small keyhole-shaped inlet recesses formed into a representative peripheral edge region and showing a representative attachment member (haptic) having two small, mating haptic-to-optic attachment protrusions dovetailed into the two optic inlet recesses;

FIG. 4 is a partial plan view similar to FIG. 1 of a multi-piece intraocular lens (IOL), except showing an optic having three, small, side-by-side keyhole-shaped inlet recesses formed into a representative peripheral edge region and showing a representative attachment member (haptic) having three small mating haptic-to-optic attachment protrusions dovetailed into the three optic inlet recesses;

FIG. 5 is a plan view of a circular modified haptic blank material, showing a central cutout corresponding to an outline an associated optic, but showing three small, side-by side haptic protrusions left remaining at opposite sides of the cutout;

FIG. 6 is a plan view of a modified optic blank material, showing an associated optic having three small, side-by-side keyhole-shaped inlet recesses formed in each of opposite peripheral edge regions, matching in size, shape and location the two sets of three small, side-by-side haptic protrusions left at the central cutout of FIG. 5;

FIG. 7 is a plan view showing the modified optic blank of FIG. 6 mated with the modified haptic blank of FIG. 5, showing the two sets of three haptic small protrusions dovetailed into the two matching sets of three optic small inlet recesses, and showing in broken lines the two corresponding haptics subsequently machined from the haptic blank so as to form a multi-piece (i.e., three-piece) intraocular lens (IOL);

FIG. 8 is a transverse cross sectional drawing taken along line 8-8 of FIG. 7, showing the relationship between the modified haptic and optic blanks before and after (in broken lines) joining of the optic and haptics into the multi-piece IOL;

FIG. 9 is a plan view, corresponding generally to FIG. 5, except showing a modified optic blank with two modified haptic blank cutouts each having three small projections made in projected haptic locations;

FIG. 10 is a plan view, corresponding generally to FIG. 6, except showing a representative modified haptic blank having three protrusions sized, shaped and located to fit into the haptic cutouts shown in the modified optic blank of FIG. 9;

FIG. 11 is a plan view corresponding generally to FIG. 7, showing the modified haptic blanks of FIG. 10 mated with the modified optic blank of FIG. 9, showing the two sets of haptic small protrusions dovetailed into the two matching sets of optic small inlet recesses, and showing in broken lines the two corresponding haptics subsequently machined from the two haptic blanks and the optic subsequently machined from the optic blank so as to form a multi-piece (i.e., three-piece) intraocular lens (IOL); and

FIG. 12 is a transverse cross sectional drawing, corresponding generally to FIG. 8, taken along line 12-12 of FIG. 11, showing the relationship between the modified haptic and optic blanks before and after (in broken lines) joining of the optic and haptics into the multi-piece IOL.

In the various FIGS. The same elements and features are given the same reference numbers and corresponding elements and features are given the original reference numbers followed by an “a”, “b”, “c” and so on as appropriate.

### DESCRIPTION OF THE PREFERRED EMBODIMENT

There is shown in the plan view of FIG. 1 portions of a multi-piece intraocular lens (IOL) 100 having a haptic blank 110 and an image-forming optic 120. Haptic blanks 110 are used, as described below, for providing haptics for maintaining an optical axis of the optic centered along the optical axis of the IOL wearer's eye. Multi-piece IOL 100 can be a posterior chamber IOL or an accommodative IOL with single or dual-lens construction (an accommodative IOL differs from a non-accommodative IOL in that it is designed to move inside the eye or change optic shape or incorporate both actions responsive to ciliary muscle contraction in the wearer's eye). Multi-piece IOL can be anterior chamber lens, for instance, an iris-fixated IOL or an angle-supported IOL.

Haptic blank 110 is formed having a single, small, haptic-to-optic attachment protrusion or protuberance 130 projecting from a haptic blank edge region 132. Haptic protrusion 130 is shown dovetailed into a small mating inlet recess 140 formed into a peripheral edge 142 of optic 120. Optic inlet recess 140 can be tear-shaped or keyhole-shaped, being enlarged in width toward an optic optical axis 144 or close to the optic peripheral edge 142so as to provide a mechanical dovetailing or locking of haptic protrusion 130 into the optic. Optic inlet recess 140 has an inlet length, 1, which is preferably between about 0.3 mm and about 0.75 mm, and a width, w, which is preferably between about 0.1 mm and about 0.25 mm, where the length is defined along the optic radial direction and the width is defined perpendicular to the optic radial direction. Haptic protrusion 10 has the same length, 1, and width, w.

It will be appreciated that haptic blank 110 would be subsequently cut (for example milled) to provide a generally conventional optic attachment haptic (not shown). It will also be appreciated that IOL 100 is preferably symmetrical with optic 120 having an inlet recess corresponding to inlet recess 140 in an optic location opposite to the shown inlet recess, and that a second haptic blank 130 would be provided with a protrusion 130 corresponding to the shown protrusion.

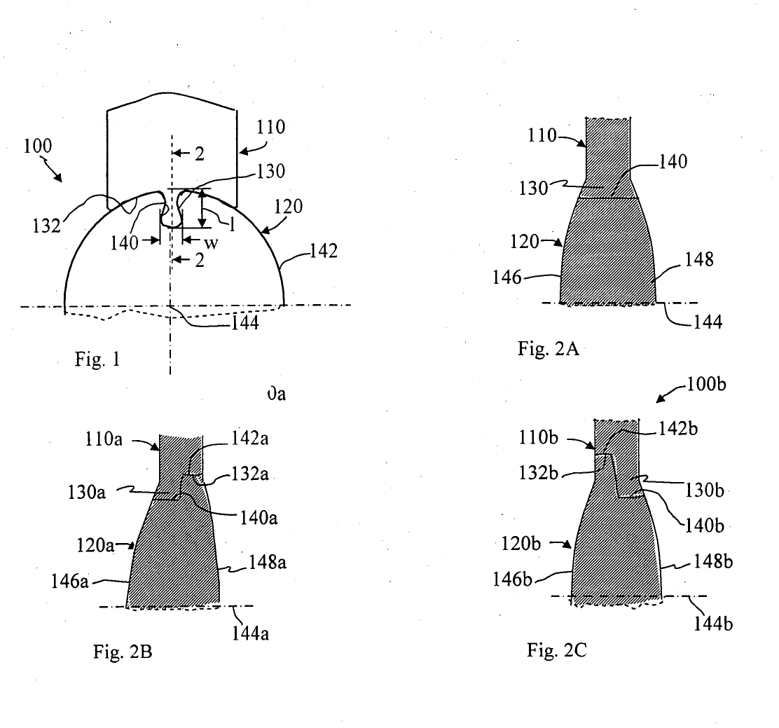

In FIG. 2A, which is a transverse cross section of IOL 100 depicted in FIG. 1, optic inlet recess 140 preferably extends entirely through optic 120 from an anterior surface 146 to a posterior surface 148. Haptic projection 130 is shown received entirely into optic recess 140.

A less preferred, first variation IOL 100a is depicted in FIG. 2B, which corresponds to cross sectional drawing of IOL 100 in FIG. 2A. An inlet recess 140a (corresponding to inlet recess 140) is formed at an optic peripheral edge 142a downwardly into an optic 120a from an anterior optic surface optic 146a, but does not extend entirely through the optic. A haptic protrusion 130a, projecting from a haptic edge 132a, is configured to dovetail closely into optic inlet recess 140a.

A less preferred, second variation IOL 100b is depicted in FIG. 2C, which also corresponds to cross sectional drawing of IOL 100 in FIG. 2A. An inlet recess 140b (corresponding to inlet recess 140) is formed at an optic peripheral edge 142b upwardly into an optic 120b from a posterior optic surface optic 146b, but does not extend entirely through the optic. A haptic protrusion 130b, projecting from a haptic edge 132b, is configured to dovetail closely into optic inlet recess 140b.

In any case, the haptic protrusion preferably has a thickness equal to the depth of the corresponding optic inlet recess into which the optic protrusion is dovetailed.

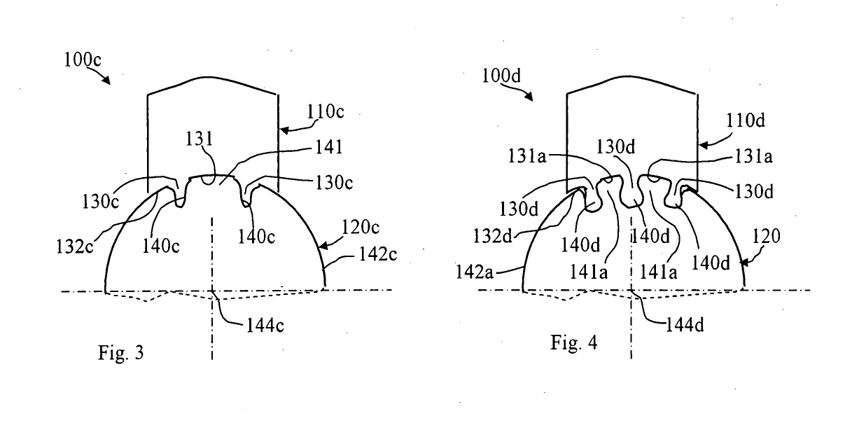

FIG. 3, which corresponds to FIG. 1 except as described below, is a partial drawing of a variation IOL 100c having an optic 120c and a haptic blank 110c. Formed into a peripheral edge 142c of optic 120c are two, side-by-side inlet recesses 140c, which are each preferably identical to above-described optic inlet recess 140. Inlet recesses 140c are preferably parallel to one another, but may alternatively be formed on a radial line from optic axis 144c.

Haptic blank 110c is formed having two side-by-side protrusions 130c, which are preferably identical to above-described haptic protrusion 130, and which are shaped and positioned to dovetail into corresponding ones of optic inlet recesses 140c, as depicted in FIG. 3.

It can also be seen from FIG. 3, that an optic peripheral edge region 141 between the two optic inlet recesses 140c comprises an optic projection or protuberance, and a haptic edge region 131 between the two haptic projections 130c comprises a haptic inlet recess, in which case optic projection 141 is received into haptic inlet recess 131 for optic-to-haptic assembly.

It will be appreciated that haptic blank 110c would be subsequently cut (for example milled) to provide a generally conventional optic attachment haptic (not shown). It will also be appreciated that IOL 100c is preferably symmetrical with optic 120c having a pair of inlet recesses corresponding to the pair of inlet recesses 140c in an optic location opposite to the shown inlet recesses, and that a second haptic blank 130c would be provided with protrusions 130c corresponding to the shown protrusions.

FIG. 4, which also corresponds to FIG. 1 except as described below, is a partial drawing of a variation IOL 100d shown having an optic 120d and a haptic blank 110d. Formed into a peripheral edge 142d of optic 120d are three, side-by-side inlet recesses 140d, for which one of the recesses is at the center along the radial meridian of the optic 120d and other two are at opposite sides of the central recess and closely parallel to it. The two side inlet recesses 140d may be similar or different from the central recess. This configuration is particularly beneficial in case of optic folding around the haptic-to-optic dovetail connection by further improving reliability of the connection. Alternatively, optic inlet recesses 140d may each be identical to above-described optic inlet recess 140.

Haptic blank 110d is formed having three side-by-side protrusions 130d, which are preferably identical to above-described haptic protrusion 130, and which are shaped and positioned to dovetail into corresponding ones of optic inlet recesses 140d, as depicted in FIG. 4. It will be appreciated that haptic blank 110d would be subsequently cut (for example milled) to provide a generally conventional optic attachment haptic (not shown). It will also be appreciated that IOL 100d is preferably symmetrical with optic 120d having a pair of inlet recesses corresponding to the pair of inlet recesses 140d in an optic location opposite to the shown inlet recesses, and that a second haptic blank 130d would be provided with protrusions 130c corresponding to the shown protrusions.

It can also be seen from FIG. 4, that two optic peripheral edge regions 141a between the three inlet recesses 140d comprise a pair of optic projections or protuberances, and two haptic edge regions 131b between the three haptic projections 130d comprise a pair of haptic inlet recesses, in which case optic projections 141a are received into haptic inlet recesses 131a for optic-to-haptic assembly.

Fixation of a complex-shaped haptic to an optic is a very tedious process due to the very small and delicate nature of the haptics. In order to improve the efficacy and accuracy of the corresponding multi-piece IOL fabrication, the present invention offers a method of making haptic-to-optic assembly at the blank fabrication step, i.e. producing a multi-material blank for multi-piece IOL fabrication.

Methods for Manufacturing Multi-Piece IOLs:

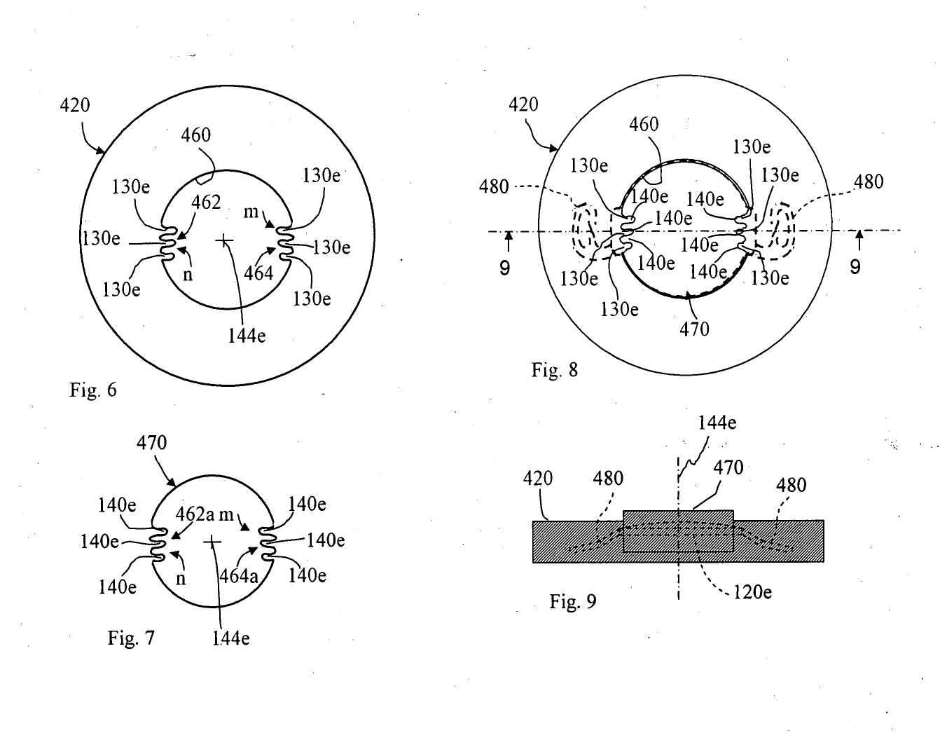

A first method for fabricating above-described multi-piece (i.e., three-piece) IOLs, in particular IOL 100d, of the present invention is illustrated in FIGS. 6-9. Shown in FIG. 6 is a circular modified haptic blank 420, which may, for example, be constructed from polymethyl methacrylate (PMMA), a hard plastic material. A central region 450 of haptic blank 420 is removed, for example, cut out by an automated milling process, around an inner periphery 460 in the size and shape of an associated IOL optic blank 470 (shown in FIG. 7). Accordingly periphery 460 has a first peripheral region 462 with cutouts for “n” (with “n” equaling three, as shown in FIG. 6) haptic protrusions 130e, and a second, opposing peripheral edge region 464 with cutouts for “m” (with “m” equal to “n” and equal to three as shown) haptic protrusions 130e. Thus, two opposing sets of “n” (three) and “m” (three) haptic protrusions remain in central region 450 after the central region has otherwise been removed.

Subsequently, a modified optic blank 470, shown in FIG. 7, is formed of a material different from the material used to fabricate haptic modified blank 420; for example, from an elastomeric material, such as a silicone or acrylic plastic material. Optic blank 470, is cast molded or cryogenically milled to a shape fitting exactly into haptic blank central region 450 cutout periphery 460, as depicted in FIG. 8. Accordingly, optic blank 470 is formed having “n” (three) inlet recesses 140e in one peripheral edge region 462a and having “m” (three) inlet recesses 140e in an opposite peripheral edge region 464a.

Upon such assembly of optic blank 470 into haptic blank 420 (FIG. 8), haptic protrusions 130e dovetail into optic recesses 140e and are cemented therein to provide positive interlocking of the haptic and optic blanks to form a single blank of dual material with the periphery made for the haptics and the central part made for the optic. The transition between one material and the other is practically instantaneous because the thickness of the applied glue is extremely thin.

Optic 120e is then cryogenically milled or lathed from optic blank 470 (FIG. 9), and opposing loop-type (or other shaped) haptics 480 (shown in broken lines in FIGS. 8 and 9) are milled or lathed from haptic blank 420, representative IOL 100d being thereby formed.

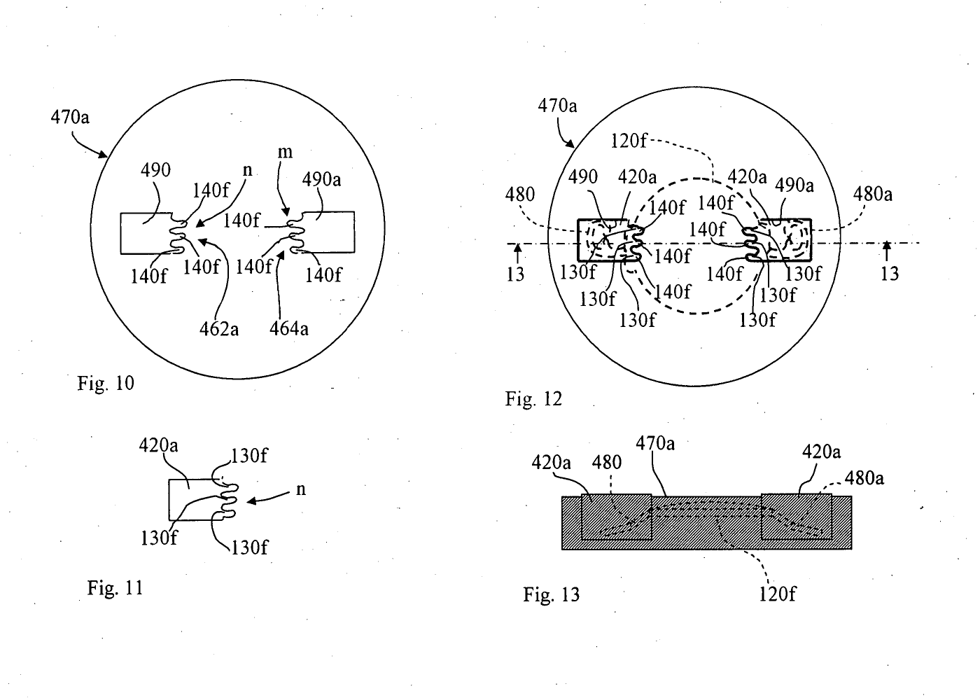

A second method for manufacturing multi-piece (i.e., three-piece) IOLs of the present invention is illustrated in FIGS. 10-13. Shown in FIG. 6 is a circular modified optic blank 470a, which may, for example, be constructed from an elastomeric material, such as a silicone or acrylic plastic material. Milled out of optic blank 470a are two opposing recesses 490 and 490a, each shaped and located to closely receive a haptic modified blank 420a, shown in FIG. 11 having “n” (three) protuberances 130f. Thus, recess 490 is formed having “n” (three) optic inlet recesses 140f in a region 462a, and recess 490a is formed having “m” (three) optic inlet recesses 140f in a region 464a (FIG. 10).

Modified haptic blank 420a, which is representative of both haptic blanks made for fitting into both optic blank recesses 490 and 490a, is preferably constructed from a material different from the material used to fabricate the optic modified blank, for example, polymethyl methacrylate (PMMA), a hard plastic material.

Upon subsequent assembly of a haptic blank 420a into each of optic blank recesses 490 and 490a, (FIG. 12), haptic protrusions 130f dovetail into corresponding optic recesses 140f, and are cemented therein to provide positive interlocking of the haptic and optic blanks. Optic blank 470a is then cryogenically milled or lathed to form optic 120f and opposing haptic blanks 420a are milled to form loop-type (or other shaped) haptics 480 and 480a (shown in broken lines in FIGS. 12 and 13), representative IOL 100d being thereby formed.

This second manufacturing method offers even more flexibility by allowing each haptic of a pair of haptics to be attached to the optic to be formed of a different material. In addition, centration of the optic over the multi-material blank is not as critical as in the first method because the whole blank is primarily made of the optic material.

Although the multi-piece IOL manufacturing methods of FIGS. 6-9 and 11-13 show haptic-optic dovetailing of two sets of three corresponding haptic protuberances and optic inlet recesses as depicted for IOL 100d of FIG. 4, it is to be understood that the methods are equally applicable haptic-optic dovetailing of two sets of two or one corresponding haptic protuberances and optic inlet recesses as depicted for IOLs 100 of FIG. 1 or 100c of FIG. 3. It will be appreciated, however, that the use of two dovetail haptic-to-optic connections provides more stability against haptic rotation and tilt relative to the optic, and the use of three dovetail haptic-to-optic connections is more robust to optic bending.

A Positional hole or holes (not shown) can be also incorporated in either above-described methods of manufacture to control positioning of the modified blanks assembly into the dual material blank.

Moreover, although the numbers “n” and “m” have been described as equal to each other, as would normally be the case, the present invention is not limited to such equality. Nor is the present invention limited to multi-piece IOLs comprising an optic having only two haptics, as depicted in FIGS. 8 and 12—in some cases, three or even four haptics may be attached to a single optic.

Referring back to FIG. 4, recall that that the two optic peripheral edge regions 141b between the three inlet recesses 140d comprise a pair of optic projections or protuberances, and two haptic edge regions 131b between the three haptic projections 130d comprise a pair of haptic inlet recesses, in which case optic projections 141a are received into haptic inlet recesses 131a for optic-to-haptic assembly. The foregoing applies to the manufacturing methods described above.

Thus, although there is described and illustrated herein various multi-piece IOL configurations and methods of manufacturing multi-piece IOLs for purposes of illustrating the manner in which the present invention may be used to advantage, it is to be understood that the invention is not limited thereto. Consequently, any and all variations and equivalent arrangements which may occur to those skilled in the applicable art are to be considered to be within the scope and spirit of the invention as set forth in the claims which are appended hereto as part of this application.

1. A multi-piece intraocular lens, said multi-piece intraocular lens comprising:
a. an optic having an optical axis and a peripheral edge, said optic peripheral edge having at least one small inlet; and
b. a haptic having an optic attachment region with at least one first small projection shaped to fit closely into said at least one optic small inlet recess through one of an optic anterior surface, an optic posterior surface and an optic peripheral edge.

2. The multi-piece intraocular lens as claimed in claim 1, wherein said optic peripheral edge has a first region with “n” first small inlet recesses and a second peripheral edge region with “m” second small inlet recesses; wherein said haptic has an optic attachment region with “n” small projections shaped to dovetail into said optic “n” first small inlet recesses; and including a second haptic having an optic attachment region with “m” small projections shaped to dovetail into said optic “m” second small inlet recesses.

3. The multi-piece intraocular lens as claimed in claim 2, wherein the numbers “n” and “m” are each selected from a group of numbers consisting of 1, 2 and 3.

4. The multi-piece intraocular lens as claimed in claim 2, wherein said first and second peripheral edge regions are located diametrically opposite of each other.

5. The multi-piece intraocular lens as claimed in claim 1, wherein said optic small inlet recess has an enlarged region towards said optical axis so as to form a general keyhole shape.

6. The multi-piece intraocular lens as claimed in claim 1, wherein said haptic is constructed from a material that is different from a material from which said optic is constructed.

7. A multi-piece intraocular lens, said multi-piece intraocular lens comprising:
a. an optic having an optical axis and a peripheral edge having at least one first small projection;
and;
b. a haptic having an optic attachment region with at least one first small inlet recess shaped to receive said at least one optic small projection.

8. The multi-piece intraocular lens as claimed in claim 7, wherein said optic peripheral edge has a first region with “n” first small projections and a second peripheral edge region with “m” second small projections; wherein said haptic has an optic attachment region with “n” small inlet recesses shaped to closely receive said optic “n” first small projections; and including a second haptic having an optic attachment region with “m” small inlet recesses shaped to closely receive said optic “m” second small projections.

9. The multi-piece intraocular lens as claimed in claim 7, wherein said haptic inlet recess has an enlarged region so as to form a general keyhole shape.

10. A multi-piece intraocular lens, said multi-piece intraocular lens comprising:
a. an optic having an optical axis and a peripheral edge, said optic peripheral edge having a first region with “n” first small inlet recesses and a second region opposite to said first region, said second region having “n” second small inlet recesses, each of said “n” first and second small inlet recesses having a general keyhole shape, the number “n” being selected from a group of numbers consisting of 1, 2 and 3;
b. a first haptic having an optic attachment region with “n” small projections shaped and located to dovetail into said optic “n” first small inlet recesses; and
c. a second haptic having an optic attachment region with “n” projections shaped and located to dovetail into said optic “n” second small inlet recesses.

11. The multi-piece intraocular lens as claimed in claim 10, wherein said first and second haptics are formed of a rigid plastic material and said optic is formed of an elastomeric plastic material.

12. A method of constructing a multi-piece intraocular lens, said method comprising the steps of:
a. forming an optic of one material with a small inlet recess in a peripheral edge region;
b. forming a positioning haptic of a different material with a small protrusion extending from an optic attachment region, said protrusion matching the shape of said optic small inlet recess; and
c. inserting said haptic protrusion into said optic inlet recess through one of an optic anterior surface, an optic posterior surface and an optic peripheral edge so as to interlock the haptic and optic together.

13. The method as claimed in claim 12, including the step of cementing said haptic protrusion into said optic inlet recess.

14. The method as claimed in claim 12, wherein the step of forming said haptic includes forming the haptic of a rigid plastic material and said step of forming said optic includes forming the optic of an elastomeric plastic material.

15. A method of constructing a multi-piece intraocular lens, said method comprising the steps of:
a. forming an optic of an elastomeric plastic material with a plurality of like small inlet recesses in each of two opposite peripheral edge regions;
b. forming first and second positioning haptics of a hard plastic material each with a plurality of small protrusions extending from an optic attachment region, said plurality of protrusions matching the shape and location of said optic small inlet recesses;
c. inserting said first haptic plurality of small protrusions into the plurality of optic small inlet recesses in one optic peripheral edge region and said second haptic plurality of small protrusions into the plurality of optic small inlet recesses in the opposite optic peripheral edge region so as to dovetail interlock the haptics and optic together; and
d. cementing said plurality of protrusions of said first and second haptics into corresponding pluralities of inlet recesses in said optic to form a composite, multi-piece intraocular lens.

16. A method of constructing a multi-piece intraocular lens, said method comprising the steps of:
a. forming a first modified blank of one material to be used for haptic fabrication with a central optic cut-out leaving at least one small protrusion for haptic to optic attachment;
b. forming a second modified blank of another material to be used for optic fabrication with at least one peripheral edge small inlet recess at a location corresponding to a location of the protrusion in said first modified blank and matching the shape of said protrusion;
c. assembling said first and second modified blanks into an interlocking, dual-material blank with said haptic small protrusion dovetailed into said optic small inlet recess; and
d. fabricating a composite, multi-piece IOL from said dual-material blank by forming a haptic in said first modified blank.

17. The method as claimed in claim 16, wherein the fabricating step includes cementing the haptic protrusion into the optic inlet recess.

18. The method as claimed in claim 16, wherein the steps of forming a haptic in said first modified blank includes forming the haptic of a rigid plastic material and said step of forming an optic in said second modified blank includes forming the optic of an elastomeric plastic material.

19. A method of constructing a multi-piece intraocular lens, said method comprising the steps of:
a. forming a first modified blank of one material to be used for haptic fabrication with a central optic cut-out leaving at least one small dovetail inlet recess in each of opposite peripheral optic cut-out regions for haptic to optic attachment;
b. forming a second modified blank of another material to be used for optic fabrication with at least one peripheral edge small protrusion in each of opposite peripheral edge regions at locations corresponding to location of the haptic inlet recesses in the first modified blank and matching the shape of said inlet recesses;
c. assembling said first and second modified blanks into a composite, dual-material blank with said optic protrusions interlockingly dovetailed and cemented into corresponding ones of said haptic inlet recesses; and
d. fabricating a composite, multi-piece IOL from said dual-material blank by forming first and second haptics in said first modified blank, including cementing the optic protrusions into the haptic inlet recesses.

20. The method as claimed in claim 19, wherein the steps of forming said first and second haptics includes forming the haptics of a rigid plastic material and said step of forming said optic includes forming the optic of an elastomeric plastic material.

21. A method of constructing a multi-piece intraocular lens, said method comprising the steps of:
a. forming a haptic from a first material, with at least one small inlet recess in an edge of said haptic;
b. forming an optic from a second material, with at least one peripheral edge small protuberance at a peripheral edge region, said at least one protuberance matching the at least one haptic small inlet recesses in shape, number and position; and
c. dovetailing and cementing said at least one optic protrusion into said at least one haptic inlet recess so as to provide a multi-piece intraocular lens.

22. The method as claimed in claim 21, wherein the steps of forming said haptic includes forming the haptic of a rigid plastic material and said step of forming said optic includes forming the optic of an elastomeric plastic material.
